SEQUENCE LISTING (2 sequences)

SEQ ID NO: 1 - protein, 655 aa, synthetic construct
MGRAMVARLGLGLLLLALLLPTQIYCNQTSVAPFPGNQNISASPNPSNATTRGAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYSSHWCCKKEVQETRRERRRLMSMEMD

SEQ ID NO: 2 - protein, 928 aa, synthetic construct
MGRLASRPLLLALLSLALCRGRVVRLEKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSQNAGFVKSPMSETKLTGDAFELYCDVVGSPTPEIQWWYAEVNRAESFRQLWDGARKRRVTVNTAYGSNGVSVLRITRLTLEDSGTYECRASNDPKRNDLRQNPSITWIRAQATISVLQKPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYSSHWCCKKEVQETRRERRRLMSMEMD

(19) United States
(12) Patent Application Publication
(10) Pub. No.: US 2026/0102356 A1
(43) Pub. Date: Apr. 16, 2026

(54) METHOD FOR LOADING DIMERIC CD24 INTO HEK293 CELL EXTRACELLULAR VESICLES WITH ADAM10 GENE KNOCKED OUT

(71) Applicant: Exocure Sweden AB, Göteborg (SE)

(72) Inventors: Yi WANG, Beijing (CN); Jialei YANG, Beijing (CN); Yanan DONG, Beijing (CN); Miaomiao XUE, Beijing (CN); Jun LI, Beijing (CN); Danfeng WANG, Beijing (CN); Wei ZHOU, Beijing (CN)

(73) Assignee: Exocure Sweden AB, Göteborg (SE)

(21) Appl. No.: 19/117,082

(22) PCT Filed: Sep. 30, 2022
(86) PCT No.: PCT/CN2022/123202

§ 371 Date: Mar. 31, 2025

(51) Int. Cl. A61K 9/50; A61K 38/00; A61P 11/00; C07K 14/47; C07K 14/705; C07K 14/775; C12N 5/071; C12N 9/22; C12N 15/11; C12N 15/86

(52) U.S. Cl. CPC A61K 9/5068; A61K 9/5089; A61P 11/00; C07K 14/4703; C07K 14/70596; C07K 14/775; C12N 5/0686; C12N 9/222; C12N 15/111; C12N 15/86; A61K 38/00; C12N 2310/20; C12N 2510/00; C12N 2740/15043

(57) ABSTRACT

The present disclosure relates to a method for loading a dimeric CD24 into an HEK293 cell exosome with ADAM10 gene knocked out. By means of loading a dimeric CD24 and/or ApoE protein into an HEK293 cell exosome with ADAM10 gene knocked out, the efficacy is improved over 1000 times compared with that of a free CD24-Fc fusion protein and ApoE protein. Meanwhile, a MyD88 inhibitor polypeptide is loaded into the exosome, so that the inhibition efficacy of the exosome on an inherent immune inflammatory response is improved. After the CD24-exosome, the ApoE-exosome, or the CD24-ApoE-exosome loaded with the described inhibitor polypeptide is phagocytosed and removed by an immune cell, the MyD88 inhibitor polypeptide can be released in the cell, the inflammation inhibition efficacy is continuously exerted, and the duration of drug's action is effectively prolonged.

### CROSS-REFERENCE TO RELATED APPLICATION

This is a 371 application of the International PCT application serial no. PCT/CN2022/123202, filed on Sep. 30, 2022. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Mar. 28, 2025, is named 155365-US-Sequence Listing and is 4,250 bytes in size.

### TECHNICAL FIELD

The present disclosure relates to the technical field of exosome loading, and specifically relates to loading dimeric CD24 into exosomes for amplifying CD24 biological effects and use thereof in the treatment of a severe inflammatory disease, as well as loading ApoE and/or MyD88 inhibitor polypeptides into CD24-loaded exosomes for multi-module synergy and use thereof in the treatment of a severe inflammatory disease.

### RELATED ART

CD24 is a highly sialylated protein molecule anchored to the cell membrane via glycosylphosphatidylinositol (GPI). CD24 is expressed on various cells and exerts immunosuppressive biological effects, such as inhibition of T-cell activation, induction of neutrophil apoptosis, inhibition of B1 cell maturation, and inhibition of macrophage/monocyte inflammatory responses. Tumor cells express CD24 as a “Don't eat me” signal to achieve immune escape. Therefore, in the field of anti-tumor therapy, antagonism of CD24 by monoclonal antibodies can indirectly activate tumor-associated macrophages to achieve anti-tumor effects. Conversely, systemic or local administration of CD24 is useful in effectively treating diseases caused by excessive immune responses, for example, the use of CD24-Fc fusion proteins in the treatment of graft-versus-host disease (GVHD).

Extracellular vesicles (EVs) have a vesicular structure and are secreted during cell growth. They often carry intracellular proteins and nucleic acids during their formation and carry extracellular proteins on or outside of the vesicle membrane, similar to the cell membrane. The variety of proteins on the membrane of extracellular vesicles is so great that extracellular vesicles can bind to and be endocytosed by almost all cells to varying degrees.

Apolipoprotein E (ApoE) is a soluble protein secreted by brain tissue and the liver, and plays an important physiological role in energy metabolism homeostasis and innate immune regulation. ApoE plays an important regulatory and ameliorative role in severe inflammatory diseases such as acute brain injuries (e.g., stroke, traumatic brain injury), chronic brain injuries (e.g., Parkinson's disease, Alzheimer's disease), acute respiratory distress syndrome, and sepsis.

MyD88 (Myeloid differentiation primary response 88) is a pivotal molecule, connecting intracellular signals from surface receptors on various immune cells to intracellular downstream signal transduction. It mediates intracellular signaling of important inflammatory receptors such as Toll-like receptors (TLRs) and interleukin-1 receptors (IL1Rs), with downstream molecules including NF-κB. MyD88 is an important target in the inhibition of innate immune responses. However, due to its primary mediation of protein-protein interactions and lack of small molecule binding pockets in its structure, small molecule inhibitors of MyD88 have been challenging to develop for decades. Loiarro et al. (DOI: 10.1074/jbc.C400613200) demonstrated in 2005 that a series of polypeptide molecules can effectively inhibit MyD88 activity. However, due to poor bioavailability and insufficient ability to enter into cells, these polypeptides are not suitable for drug development.

Existing technologies enable the collection and purification of exosomes containing CD24 (Exo-CD24) from HEK293 cells overexpressing CD24 for inflammatory treatment. However, the efficacy of CD24 molecules loaded on exosomes through only overexpression have no obvious advantages compared to CD24-Fc fusion proteins. Moreover it has a limited drugability due to the more complex production process and higher production cost compared to CD24-Fc fusion proteins. Besides, MyD88 inhibitor polypeptides have a short elimination half-life in vivo and require cell membrane penetration to be effective, with low bioavailability. Fusion proteins formed by MyD88 inhibitor polypeptides and membrane-penetrating peptides lack targeting property and pose safety risks due to extensive systemic immunosuppression. In addition, the full-length ApoE protein usually exists in the form of lipoprotein, with the free ApoE protein or its mimetic polypeptide exhibiting weak efficacy and poor in vivo stability.

Therefore, the present disclosure aims at loading CD24, ApoE, MyD88 inhibitory polypeptides, or a combination thereof by using extracellular vesicles/exosomes as carriers to achieve the treatment of diseases related to a severe inflammatory response.

### SUMMARY OF INVENTION

In the present disclosure, dimeric CD24 and/or ApoE protein is loaded into exosomes from HEK293 cells with ADAM10 gene knockout (the technology of protein loading into exosomes and dimer protein loading sequence are known in the prior art, referenced in patent: 202210549553.7), achieving over a 1000-fold enhancement in efficacy compared to free CD24-Fc fusion protein and ApoE protein. Additionally, MyD88 inhibitor polypeptide is loaded into these exosomes (the technology of polypeptide loading is referenced in patent: 202111263036.5), enhancing the exosomes' inhibitory efficacy on inflammatory responses.

The present disclosure provides a method for loading dimeric CD24 into exosomes from HEK293 cells with ADAM10 gene knocked out, including steps of:
(1) knocking out ADAM10 gene in HEK293 cells,
(2) loading dimeric CD24 and/or dimeric ApoE into exosomes from HEK293 cells with ADAM10 gene knockout, and
(3) verifying loading amount and/or function.

Further, in step (1), the knockout of ADAM10 gene in HEK293 cells is carried out using a CRISPR gene editing system or method.

Further, in step (2), the loading includes constructing pCD24-Fc-Ig3(NPTN-Ig3)-TMD(NPTN-TMD)-ICD(EWI-F-ICD) plasmid using VB220306-1137jmq as a vector, co-transfecting with Sleeping Beauty transposase expression vector pCMV-(CAT)T7-SB100X into or infecting HEK293 cells and/or ADAM10⁻ HEK293 cells via a viral vector with the plasmid, and screening a single clone to obtain an engineered stable transgenic cell line of HEK293-CD24 or ADAM10⁻ HEK293-CD24.

Further, step (2) includes expanding and culturing an engineered stable transgenic cell line to a cell density of 5E+06 cells/mL, performing centrifugation at 5000 rpm for 30 min, and performing purification from cell supernatant to obtain engineered dimeric CD24 loaded-exosomes.

Further, in step (3), the verification of loading amount is carried out by collecting cellular proteins for Western blot assay.

Further, in step (3), the verification of function is carried out by assessing a role of dimeric CD24 loaded-exosomes on mortality and liver integrity in a mouse model of acute liver failure.

Further, step (2) includes loading dimeric ApoE protein into exosomes.

Further, the loading of dimeric ApoE protein into exosomes includes constructing pApoE-Fc-Ig3(NPTN-Ig3)-TMD(NPTN-TMD)-ICD(EWI-F-ICD) plasmid with pSLenti-CMV--PGK-PuroWPRE as a vector, infecting ADAM10⁻ HEK293 cells at an MOI of 10, replacing medium after 16 h and 24 h of infection, and screening a single clone to obtain an engineered stable transgenic cell line of ADAM10⁻ HEK293-ApoE.

Further, step (2) includes loading dimeric CD24 and dimeric ApoE protein into exosomes.

Further, the loading of dimeric CD24 and dimeric ApoE protein into exosomes includes co-transfecting into or infecting HEK293 cells via a viral vector with a constructed pApoE-Fc-Ig3(NPTN-Ig3)-TMD(NPTN-TMD)-ICD(EWI-F-ICD) plasmid and a constructed pCD24-Fc-Ig3(NPTN-Ig3)-TMD(NPTN-TMD)-ICD(EWI-F-ICD) plasmid, and screening to obtain an engineered stable transgenic cell line of ADAM10⁻ HEK293-CD24-ApoE.

Further, the method further includes expanding and culturing an engineered stable transgenic cell line to a cell density of 5E+06 cells/mL, performing centrifugation at 5000 rpm for 30 min, and performing purification from cell supernatant to obtain engineered loaded exosomes.

Further, in step (3), the verification includes detecting an immunosuppressive activity of different EVs and proteins using a PBMC inflammation model and/or establishing a mouse model of acute liver failure, and verifying a hepatoprotective effect of CD24 EV based on mortality.

Further, step (2) includes loading both dimeric CD24 and MyD88 inhibitor polypeptide into exosomes.

Further, the loading of both dimeric CD24 and MyD88 inhibitor polypeptide into exosomes includes mixing dimeric CD24 exosomes with MyD88 inhibitor polypeptide, adjusting pH to 8.0, purifying with Capto core700 to remove free MyD88 inhibitor polypeptide, and collecting a flow-through to obtain CD24 exosomes loaded with MyD88 inhibitor polypeptide.

Further, the dimeric CD24 exosomes are mixed with the MyD88 inhibitor polypeptide at a ratio of 1 mg of MyD88 inhibitor polypeptide per 1E+12 exosome particles.

Further, in step (3), the verification includes detecting an immunosuppressive activity of different EVs and proteins using a PBMC inflammation model.

Further, in step (3), the verification includes detecting mortality in a mouse model of sepsis.

Further, step (2) includes loading dimeric CD24, dimeric ApoE and MyD88 inhibitor polypeptide into exosomes.

Further, in step (3), the verification includes detecting a significant reduction in mortality and protection of lung tissue integrity in a mouse model of acute respiratory distress syndrome.

Further, the present disclosure provides an exosome produced by the method above.

Further, the present disclosure provides use of the exosome above in the manufacturing of a medicament.

Further, the medicament is useful in the treatment of an inflammation.

The present disclosure further provides a pharmaceutical composition including the exosome of the present disclosure.

The present disclosure further provides a method of treatment, including administering the pharmaceutical composition of the present disclosure.

### Beneficial Effects

By loading dimeric CD24 and/or ApoE protein into exosomes from HEK293 cells with ADAM10 gene knockout, and further loading MyD88 inhibitor polypeptide into these exosomes, the present disclosure obtains CD24-exosomes, ApoE-exosomes or CD24-ApoE-exosomes loaded with MyD88 inhibitor polypeptide, with a significantly improved efficacy. The method and the product provided by the present disclosure have the following advantages: (1) Simply overexpressed CD24-loaded exosomes does not significantly enhance efficacy compared to CD24-Fc fusion proteins. However, the dimeric CD24 exosomes provided by the present disclosure require only one-thousandth to one-ten-thousandth the concentration of CD24-Fc fusion proteins to achieve the same level of inflammation suppression as the fusion proteins. In vivo efficacy studies also demonstrate that the effective dose of dimeric CD24 exosomes is very low, which not only reduces the cost of the drug per dose but also improves drug safety and druggability. (2) The inflammation suppressive efficacy of ApoE loaded in exosomes is significantly greater than that of free ApoE protein. (3) Exosomes loaded with both dimeric CD24 and ApoE protein achieve a technical effect that addresses both disease treatment and immune modulation, making them a promising focus for drug development in immune-regulating medicinal exosomes with substantial market potential. (4) Although exosome-based drugs are quickly cleared by innate immune cells, the loading of MyD88 inhibitor polypeptide allows for continued release of the MyD88 inhibitor polypeptide within the cells even after the exosomes are phagocytosed and cleared and continued inflammation inhibition effect, thereby significantly prolonging the duration of drug efficacy, and this provides exosomes loaded with both CD24/ApoE and MyD88 inhibitor polypeptide with a better drugability compared to exosomes without polypeptides.

### BRIEF DESCRIPTION OF DRAWINGS

FIG. 1 shows the Western blot results verifying the baseline expression after ADAM10 knockout in the present disclosure.

FIG. 2 shows the expression of surface proteins from exosomes derived from wild-type cells in the present disclosure.

FIG. 3 shows the expression of surface proteins from exosomes derived from cells after ADAM10 knockout in the present disclosure.

FIG. 4 shows the statistics on the anti-inflammatory activity of free CD24-Fc fusion protein, monomer CD24 loaded-EV, and dimeric CD24-loaded exosomes in an in vitro PBMC cell inflammation model in the present disclosure.

FIG. 5 shows the statistics on mouse mortality following administration of high-dose and low-dose CD24 EV in the present disclosure.

FIG. 6 shows the protective effects of CD24 EV on liver tissue in the present disclosure.

FIG. 7 shows the statistics on the anti-inflammatory activity of free ApoE protein and dimeric ApoE-loaded exosomes in an in vitro PBMC cell inflammation model in the present disclosure.

FIG. 8 shows the statistics on mouse mortality following administration of CD24 EV and CD24-ApoE EV in the present disclosure.

FIG. 9 shows the statistics on the anti-inflammatory activity of free ApoE protein, dimeric CD24 loaded-exosomes, and exosomes loaded with both dimeric CD24 and MyD88 inhibitor polypeptide in an in vitro PBMC cell inflammation model in the present disclosure.

FIG. 10 shows the statistics on mouse mortality following administration of exosomes loaded with dimeric CD24, dimeric ApoE, and MyD88 inhibitor polypeptide in a mouse model of acute respiratory distress syndrome in the present disclosure.

FIG. 11 shows the expression levels of cytokines in the blood of mice following administration of exosomes in different treatment groups in the present disclosure.

FIG. 12 shows the statistics on mouse mortality following administration of exosomes in different treatment groups in present disclosure.

### DESCRIPTION OF EMBODIMENTS

Technical solutions of embodiments of the present disclosure will be clearly and completely described below. Apparently, the embodiments described in the following are only some embodiments of the present disclosure, rather than all the embodiments. Any other embodiments obtained by those skilled in the art based on the embodiments in the present disclosure without any creative work fall within the scope of protection of the present disclosure.

### Example 1: Knockout of ADAM10 on Exosome Surface Significantly Increased Dimeric CD24 Loading on Exosome Surface

Wild-type HEK293 cells were subjected to ADAM10 knockout, resulting in the establishment of an ADAM10-KO cell line. This knockout prevented ADAM10 from cleaving membrane proteins on the exosome surface and increased the loading of proteins on the exosome surface.

### 1. Methods:

(1) ADAM10 Knockout: The ADAM10-gRNA plasmid was transfected into HEK293 cells using the CRISPR system and its specific method, after which cells were plated and the single-clone cells were picked for sequencing to obtain a cell line with successful ADAM10 knockout. The Western blot assay was carried out to confirm the absence of residual ADAM10 expression.
(2) Plasmid Design and Construction: The plasmid pCD24-Fc-Ig3(NPTN-Ig3)-TMD(NPTN-TMD)-ICD(EWI-F-ICD) was constructed using VB220306-1137jmq (Yunzhou Biotech, Sleeping Beauty Expression Vector) as the vector. The module design was referenced from the accepted patent (Patent Application No. 202210549553.7) of the Applicant's company. The amino acid sequence is shown below.

Seq1:
MGRAMVARLGLGLLLLALLLPTQIYCNQTSVAPFPGNQNISASPNPSNA
TTRGAESKYGPPCPPCPAPEAAGGPSVFLFPPKPKDTLMISRTPEVTCV
VVDVSQEDPEVQFNWYVDGVEVHNAKTKPREEQFNSTYRVVSVLTVLHQ
DWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSR
LTVDKSRWQEGNVFSCSVMHEALHNHYTQKSLSLSLGGGGGSGGGGSGG
GGSQNAGFVKSPMSETKLTGDAFELYCDVVGSPTPEIQWWYAEVNRAES
FRQLWDGARKRRVTVNTAYGSNGVSVLRITRLTLEDSGTYECRASNDPK
RNDLRQNPSITWIRAQATISVLQKPRIVTSEEVIIRDSPVLPVTLQCNL
TSSSHTLTYSYWTKNGVELSATRKNASNMEYRINKPRAEDSGEYHCVYH
FVSAPKANATIEVKAAPDITGHKRSENKNEGQDATMYCKSVGYPHPDWI
WRKKENGMPMDIVNTSGRFFIINKENYTELNIVNLQITEDPGEYECNAT
NAIGSASVVTVLRVRSHLAPLWPFLGILAEIIILVVIIVVYSSHWCCKK
EVQETRRERRRLMSMEMD

(3) Acquision of Stable Transgenic Cell Lines: The plasmid constructed in step (2) was co-transfected with the Sleeping Beauty (SB) transposase expression vector pCMV-(CAT)T7-SB100X into HEK293 or ADAM10-HEK29 cells. Single clones were screened to obtain HEK293-CD24 or ADAM10⁻ HEK293-CD24 engineered stable transgenic cell lines.
(4) Preparation of Engineered dimeric CD24 Exosomes: The engineered stable transgenic cell lines were expanded and cultured to a cell density of 5E+06 cells/mL, and centrifuged at 5000 rpm for 30 min to collect the cell supernatant. The engineered dimeric CD24 loaded-exosomes were obtained from the cell supernatant by purification.
(5) Loading Amount Verification: Exosomes purified from the cell supernatants of HEK293, ADAM10⁻ HEK293, HEK293-CD24, and ADAM10⁻ HEK293-CD24 were subjected to Western blot assay with CD24 antibody, Fc antibody, and EGFP antibody to compare the loading amount.

### 2. Results:

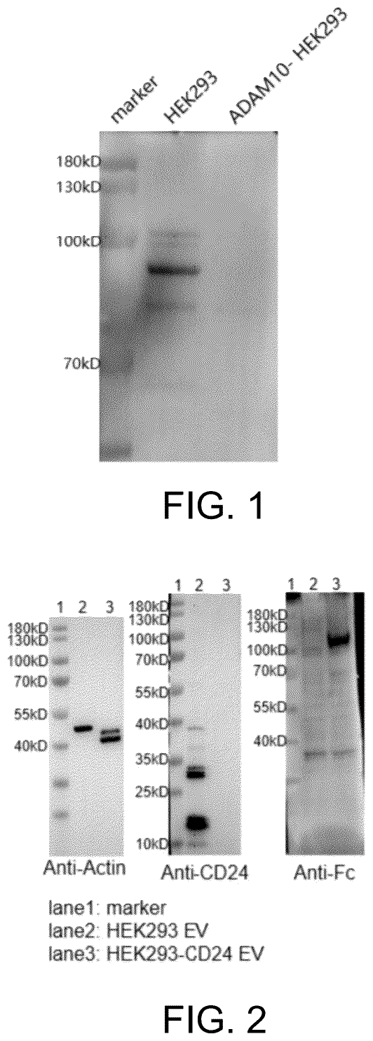

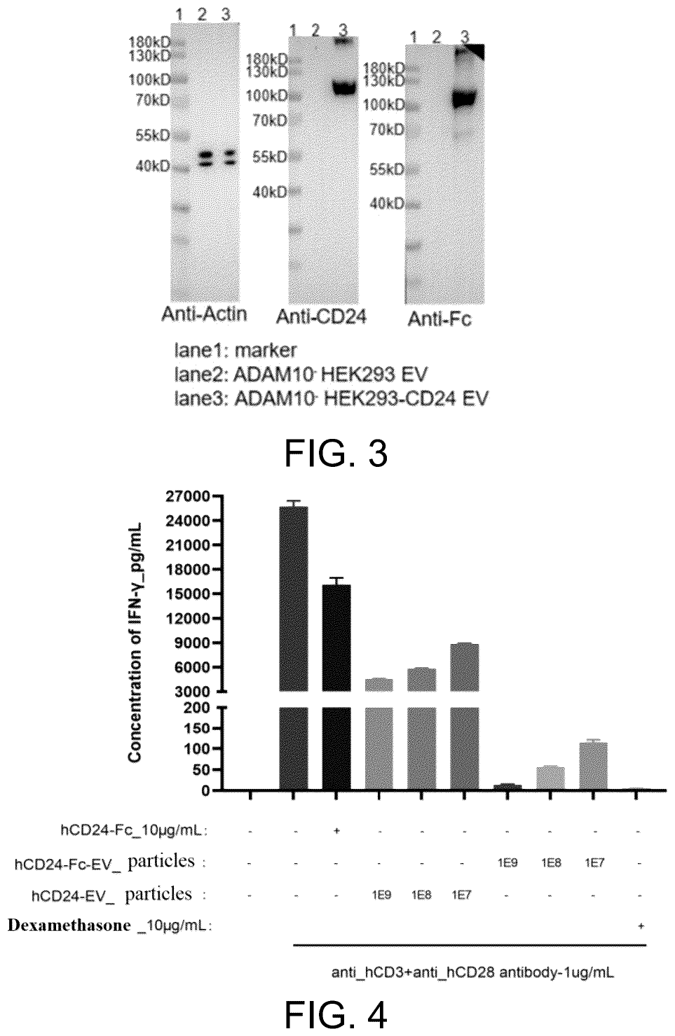

The results are shown in FIGS. 1-3.

### 3. Conclusion:

It was confirmed that ADAM10 knockout resulted in the absence of residual ADAM10 protein expression. CD24-Fc-Ig3(NPTN-Ig3)-TMD(NPTN-TMD)-ICD(EWI-F-ICD) has a theoretical molecular weight of 60 kD, and the dimer has a molecular weight of around 120 kD. The results of Western blot showed the presence of dimer proteins. CD24 on the membrane surface of wild-type cell-derived exosomes was cleaved by ADAM10, and the CD24 antibody did not detect the protein. CD24 on the surface of ADAM10⁻ cell-derived exosomes was retained, and the loading of CD24 on the exosome surface was significantly enhanced.

### Example 2: Dimeric CD24 Loaded-Exosomes Showed Thousands of Times More Potent Immunosuppressive Activity than Free CD24-Fe Fusion Protein in an In Vitro Cell Model

According to the fact that CD24 can exert immunosuppressive biological effects, such as inhibiting T cell activation, inducing neutrophil apoptosis, inhibiting B1 cell maturation, and inhibiting macrophage/monocyte inflammatory responses, the inhibitory effect of CD24-loaded exosomes (CD24-EV) on the inflammatory response of PBMCs was detected. In addition, the engineered dimeric CD24 loaded-exosomes can achieve local aggregation of CD24, leading to the amplification of biological effects, and thus exhibiting a stronger immunosuppressive activity than monomer CD24-EV.

### 1. Methods:

(1) See Example 1 for the method of preparing dimeric CD24-loaded EV.
(2) Preparation of monomer CD24-loaded EV:

Plasmid Design and Construction: The plasmid pCD24-Ig3(NPTN-Ig3)-TMD(NPTN-TMD)-ICD(EWI-F-ICD) was constructed using VB220306-1137jmq (Yunzhou Biotech, Sleeping Beauty expression vector) as the vector.

Acquisition of stable transgenic cell line: The plasmid constructed in step (1) was co-transfected into ADAM10-HEK29 cells with the Sleeping Beauty transposase expression vector pCMV-(CAT)T7-SB100X. Single clones were screened to obtain ADAM10⁻ HEK293-CD24 engineered stable transgenic cell line.

Preparation of engineered monomer CD24 exosomes: The engineered stable transgenic cell line was expanded and cultured to a cell density of 5E+06 cells/mL, and centrifuged at 5000 rpm for 30 min to collect the cell supernatant. The engineered exosomes loaded with monomer CD24 were obtained from the cell supernatant by purification. They were used for the cell assay after confirming that the loading amount was normal.

Detection of the immunosuppressive activity of different EVs and proteins in a PBMC inflammation model: PBMCs were used for the experiment. An inflammation model was established by using 1 μg/ml anti-human CD3 Antibody (coated) and 1 μg/ml anti-human CD28 Antibody (free). The positive drug (dexamethasone), free CD24-Fc protein, and various doses of engineered dimeric CD24-loaded EV and monomer CD24-loaded EV were administered for 72 h, and their inhibitory activity on the inflammatory response of PBMCs was assessed.

### 2. Results:

The results are shown in Table 1 and FIG. 4.

TABLE 1
Concentration of IFN-γ in PBMC Supernatants for Different Treatment Groups

| Anti_hCD3 + | | Corresponding | | | | | | |
|---|---|---|---|---|---|---|---|---|
| anti_hCD28 | | hCD24 Protein | | Detected IFN-γ | | | IFN-γ_pg/mL- | |
| antibody_1 μg/mL | Treatment | Concentration | | concentration (pg/mL) | | | Mean | SD |
| − | — | | | 0.23 | 0.56 | 0.34 | 0.4 | 0.17 |
| + | — | | | 26352 | 25869 | 24987 | 25736.0 | 692.15 |
| | hCD24- | | | 15210 | 16847 | 16324 | 16127.0 | 836.09 |
| | Fc_10 μg/mL | | | | | | | |
| | hCD24-EV_1E9 | 4 | μg/mL | 4527 | 4618 | 4564 | 4569.7 | 45.76 |
| | hCD24-EV_1E8 | 0.4 | μg/mL | 5892 | 5764 | 5823 | 5826.3 | 64.07 |
| | hCD24-EV_1E7 | 0.04 | μg/mL | 8963 | 8827 | 8869 | 8886.3 | 69.64 |
| | hCD24-Fc- | 4 | μg/mL | 15.6 | 12.8 | 13.4 | 13.9 | 1.47 |
| | EV_1E9 | | | | | | | |
| | hCD24-Fc- | 0.4 | μg/mL | 55.6 | 58.9 | 57.5 | 57.3 | 1.66 |
| | EV_1E8 | | | | | | | |
| | hCD24-Fc- | 0.04 | μg/mL | 108 | 121 | 116 | 115.0 | 6.56 |

| | | | | | |
|---|---|---|---|---|---|
| EV_1E7 | | | | | |
| Dexamethasone_10 | 5.62 | 5.78 | 6.51 | 6.0 | 0.47 |
| μg/mL | | | | | |

### 3. Conclusion:

Free CD24-Fc fusion protein, monomer CD24 loaded-EV and dimeric CD24 loaded-exosomes all demonstrated better inflammation inhibitory activity in the in vitro PBMC inflammation model compared to the model group. In particular, dimeric CD24 loaded-exosomes showed immunosuppressive activity that was thousands of times stronger than that of free CD24-Fc fusion proteins and hundreds of times stronger than that of monomer CD24 loaded-exosomes. The immunosuppressive activity increased with increasing doses of exosomes.

### Example 3: Dimeric CD24 Loaded-Exosomes Effectively Reduced Mortality and Maintain Liver Tissue Integrity in a Mouse Model of Acute Liver Failure

Engineered dimeric CD24 loaded-exosomes can achieve local aggregation of CD24, leading to an amplification of the biological effect. A mouse model of acute liver failure was established and the hepatoprotective effect of CD24 was verified by detection of mortality and observation of liver tissue section.

### 1. Methods:

(1) Preparation of engineered dimeric CD24 exosomes: The engineered stable transgenic cell line (same as the stable transgenic cell line in Example 1) was expanded and cultured to a cell density of 5E+06 cells/mL, and centrifuged at 5000 rpm for 30 min to collect the cell supernatant. The engineered dimeric CD24-loaded exosomes were obtained from the cell supernatant by purification.
(2) Verification of hepatoprotective effects: Eight-week-old male Balb/c mice (Beijing Vital River) were selected for the experiment. A model was established by intraperitoneal injection of 30% CCl₄ (v/v) in the dose of 5 mL/kg. Treatment was administered 2 hours after the CCl₄ injection. Mice were divided into groups to receive the positive drug (bifendate, approximately 100 μg at a dose of 5 mg/kg body weight) and various doses of CD24EV (the low-dose group received 1E7 exosome particles per mouse, and the high-dose group received 1E9 exosome particles per mouse, with each 1E9 exosomes loaded with 1 μg of CD24 protein). Three doses were given within 24 hours at time points of 4 h, 12 h, and 20 h post-modeling, and the hepatoprotective effect was assessed by observing the mortality of mice. Liver tissues from the CD24EV treatment group and the liver failure group were collected for histological analysis to assess liver tissue damage.

### 2. Results:

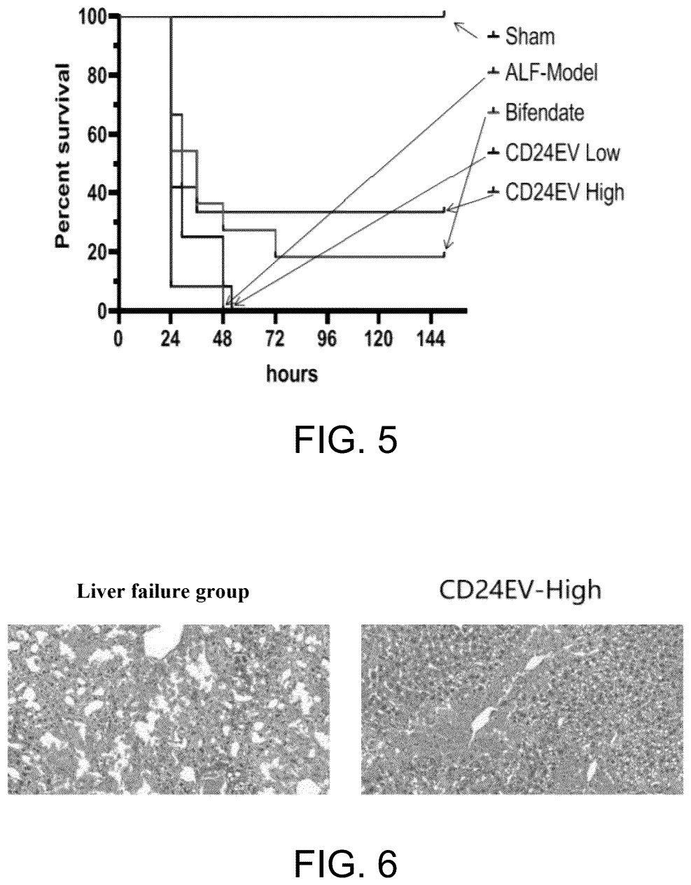

The results are shown in FIGS. 5-6.

3. Conclusion: Compared to the model group, both the high-dose and low-dose CD24-EV groups demonstrated the effect of reducing mortality, with the high-dose group showing an even lower mortality than the positive drug group. When compared to the liver failure group, the high-dose CD24 group exhibited significant protective effects on liver tissue.

### Example 4: Exosomes Loaded with Dimeric ApoE Showed a Stronger Immunosuppressive Effect in an In Vitro Cell Model Compared to Free ApoE Protein

### 1. Methods:

(1) Plasmid Design and Construction: The plasmid pApoE-Fc-Ig3(NPTN-Ig3)-TMD(NPTN-TMD)-ICD(EWI-F-ICD) was constructed using the lentiviral vector pSLenti-CMV-PGK-PuroWPRE (OBiO Technology) as the vector. The module design referenced an accepted patent (Patent Application No. 202210549553.7) of Applicant's company. The amino acid sequence is shown below.

Seq2:
MGRLASRPLLLALLSLALCRGRVVRLEKVEQAVETEPEPELRQQTEWQS
GQRWELALGRFWDYLRWVQTLSEQVQEELLSSQVTQELRALMDETMKEL
KAYKSELEEQLTPVAEETRARLSKELQAAQARLGADMEDVCGRLVQYRG
EVQAMLGQSTEELRVRLASHLRKLRKRLLRDADDLQKRLAVYQAGAREG
AERGLSAIRERLGPLVEQGRVRAATVGSLAGQPLQERAQAWGERLRARM
EEMGSRTRDRLDEVKEQVAEVRAKLEEQAQQIRLQAEAFQARLKSWFEP
LVEDMQRQWAGLVEKVQAAVGTSAAPVPSDNHAESKYGPPCPPCPAPEA
AGGPSVFLFPPKPKDTLMISRTPEVTCVVVDVSQEDPEVQFNWYVDGVE
VHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHE
ALHNHYTQKSLSLSLGGGGGSGGGGSGGGGSQNAGFVKSPMSETKLTGD
AFELYCDVVGSPTPEIQWWYAEVNRAESFRQLWDGARKRRVTVNTAYGS
NGVSVLRITRLTLEDSGTYECRASNDPKRNDLRQNPSITWIRAQATISV
LQKPRIVTSEEVIIRDSPVLPVTLQCNLTSSSHTLTYSYWTKNGVELSA
TRKNASNMEYRINKPRAEDSGEYHCVYHFVSAPKANATIEVKAAPDITG
HKRSENKNEGQDATMYCKSVGYPHPDWIWRKKENGMPMDIVNTSGRFFI
INKENYTELNIVNLQITEDPGEYECNATNAIGSASVVTVLRVRSHLAP
LWPFLGILAEIIILVVIIVVYSSHWCCKKEVQETRRERRRLMSMEMD

(2) Acquisition of stable transgenic cell line: The plasmid constructed in step (1) was packaged into viruses and used to infect ADAM10⁻ HEK293 cells according to MOI=10. The medium was replaced at 16 h and 24 h after infection, and single clones were screened to obtain the ADAM10⁻ HEK293-ApoE engineered stable transgenic cell line.
(3) Preparation of dimeric ApoE-exosomes: The engineered stable transgenic cell line was expanded and cultured to a cell density of 5E+06 cells/mL, and centrifuged at 5000 rpm for 30 min to collect the cell supernatant. The engineered exosomes loaded with dimeric ApoE were obtained from the cell supernatant by purification.

Detection of the immunosuppressive activity of different EVs and proteins in a PBMC inflammation model: PBMCs were used for the experiment. An inflammation model was established by using 1 g/ml anti-human CD3 Antibody (coated) and 1 μg/ml anti-human CD28 Antibody (free). The positive drug (dexamethasone), free ApoE protein, and various doses of engineered dimeric ApoE-loaded EV were administered for 72 h, and their inhibitory activity on the inflammatory response of PBMCs was assessed.

### 2. Results:

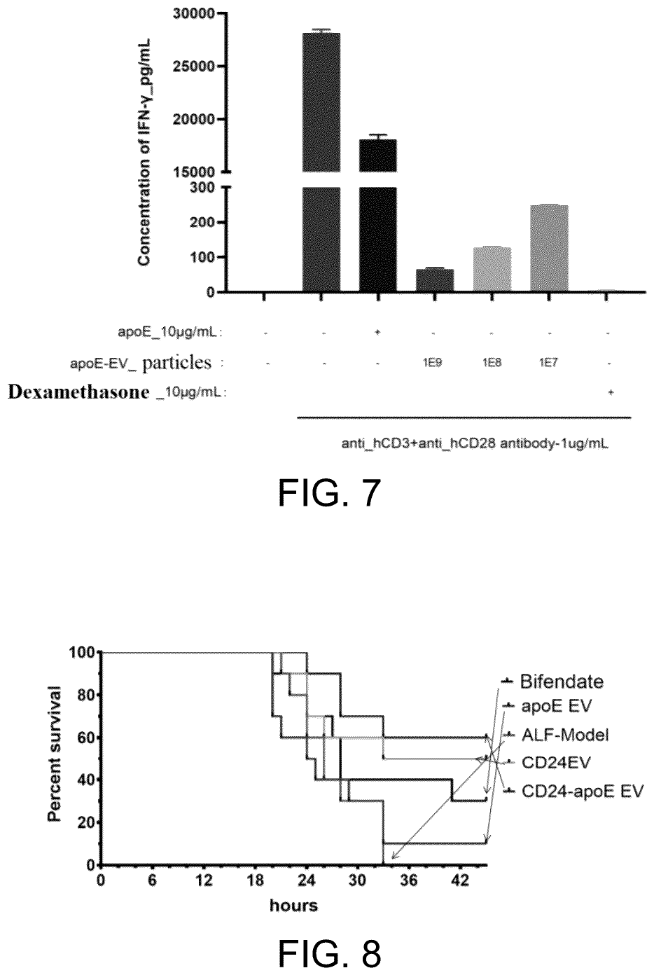

The results are shown in Table 2 and FIG. 7.

TABLE 2
Concentration of IFN-γ in PBMC Supernatants for Different Treatment Groups

| Anti_hCD3 + | | Corresponding | | | | | | |
|---|---|---|---|---|---|---|---|---|
| anti_hCD28 | | ApoE Protein | | Detected IFN-γ | | | IFN-γ_pg/mL- | |
| antibody_1 μg/mL | Treatment | Concentration | | concentration (pg/mL) | | | Mean | SD |
| − | — | | | 0.23 | 0.56 | 0.34 | 0.4 | 0.17 |
| + | — | | | 28534 | 27965 | 28012 | 28170.3 | 315.82 |
| | ApoE_10 | | | 18563 | 17689 | 18024 | 18092.0 | 440.95 |
| | μg/mL | | | | | | | |
| | ApoE- | 4 | μg/mL | 64 | 62 | 70 | 65.3 | 4.16 |
| | EV_1E9 | | | | | | | |
| | ApoE- | 0.4 | μg/mL | 130 | 128 | 125 | 127.7 | 2.52 |
| | EV_1E8 | | | | | | | |
| | ApoE- | 0.04 | μg/mL | 248 | 251 | 249 | 249.3 | 1.53 |

| | | | | | |
|---|---|---|---|---|---|
| EV_1E7 | | | | | |
| Dexamethasone_10 | 5.5 | 5.8 | 6.2 | 5.8 | 0.35 |
| μg/mL | | | | | |

3. Conclusion: Both free ApoE proteins and dimeric ApoE loaded-exosomes demonstrated better inflammation inhibitory activity in the in vitro PBMC inflammation model compared to the model group. In particular, dimeric ApoE loaded-exosomes showed immunosuppressive activity that was thousands of times stronger than that of free ApoE proteins. The immunosuppressive activity increased with increasing doses of exosomes.

### Example 5: Exosomes Loaded with Both Dimeric CD24 and Dimeric ApoE Significantly Reduced Mortality in a Mouse Model of Acute Liver Failure

Exosomes from ADAM10⁻ HEK293 cells were loaded with both dimeric CD24 and dimeric ApoE proteins. A mouse model of acute liver failure was established, and the hepatoprotective effect of CD24 was verified by assessing mouse mortality.

### 1. Methods:

(1) Plasmid Design and Construction: The plasmids pCD24-Fc-Ig3(NPTN-Ig3)-TMD(NPTN-TMD)-ICD(EWI-F-ICD) and pApoE-Fc-Ig3(NPTN-Ig3)-TMD(NPTN-TMD)-ICD(EWI-F-ICD) were constructed using VB220306-1137jmq (Yunzhou Biotech, Sleeping Beauty expression vector) as the vector.
(2) Acquisition of Stable Transgenic Cell Line: The two plasmids constructed in step (1) were co-transfected into ADAM10⁻ HEK29 cells with the Sleeping Beauty transposase expression vector pCMV-(CAT)T7-SB100X, and single clones were screened to obtain the ADAM10-HEK293-CD24-ApoE engineered stable transgenic cell line.
(3) Preparation of CD24-ApoE-Exosomes: The engineered stable transgenic cell line was expanded and cultured to a cell density of 5E+06 cells/mL, and centrifuged at 5000 rpm for 30 min to collect the cell supernatant. The engineered dimeric CD24-ApoE loaded-exosomes were obtained from the cell supernatant by purification.

Verification of hepatoprotective effects: Eight-week-old male Balb/c mice (Beijing Vital River) were selected for the experiment. A model was established by intraperitoneal injection of 30% CCl₄ (v/v) in the dose of 5 mL/kg. Treatment was administered 2 hours after the CCl₄ injection. Mice were divided into groups to receive the positive drug (bifendate, approximately 100 μg at a dose of 5 mg/kg body weight), CD24EV, ApoE EV, and CD24-ApoE EV (each group receiving 1E9 exosomes loaded with 1 μg of protein). Three doses were given within 24 hours at time points of 4 h, 12 h, and 20 h post-modeling, and the hepatoprotective effect was assessed by observing the mortality of mice.

### 2. Results:

The results are shown in FIG. 8.

3. Conclusion: The CD24 EV and CD24-ApoE EV groups exhibited lower mortality compared to the positive drug group, with the CD24-ApoE EV group demonstrating a superior effect in reducing mouse mortality compared to the group loaded with only CD24 EV.

### Example 6: Exosomes Loaded with Both Dimeric CD24 and MyD88 Inhibitor Polypeptide Significantly Suppressed Inflammatory Responses in an In Vitro Cell Model

Loading MyD88 inhibitor polypeptide into CD24-loaded exosomes enhanced their immunosuppressive efficacy against innate immune inflammatory responses. After being phagocytosed and cleared by immune cells, the CD24-exosomes loaded with MyD88 inhibitor polypeptide released the MyD88 inhibitor polypeptide inside the cells, continuing to exert anti-inflammatory effects and effectively prolonging the duration of the drug's action.

### 1. Methods:

(1) Preparation of dimeric CD24-Exosomes: The plasmid construction, cell line screening and exosome preparation processes were consistent with those in Example 1.
(2) Loading of MyD88 Inhibitor Polypeptide: dimeric CD24 exosomes were mixed well with MyD88 inhibitor polypeptide (1 mg of MyD88 added to 1E+12 exosome particles). The solution was adjusted to a pH of 8.0 (or 9.0, 10.0) with Na₂CO₃, and then purified using Capto core700 to remove free MyD88 inhibitor polypeptide. The flow-through was collected to obtain CD24 exosomes loaded with MyD88 inhibitor polypeptide. The loading amount of MyD88 inhibitor polypeptide was assessed using ELISA (peptide loading technology referenced in patent: 202111263036.5).
(3) Detection of the immunosuppressive activity of different EVs and proteins in a PBMC inflammation model: PBMCs were used for the experiment. An inflammation model was established by using 1 μg/ml anti-human CD3 Antibody (coated) and 1 μg/ml anti-human CD28 Antibody (free). The positive drug (dexamethasone), free CD24-Fc fusion protein, and various doses of engineered dimeric CD24 loaded-EV and engineered dimeric CD24+MyD88 inhibitor polypeptide loaded-EV were administered for 72 h or 120 h, and their inhibitory activity on the inflammatory response of PBMCs was assessed

### 2. Results:

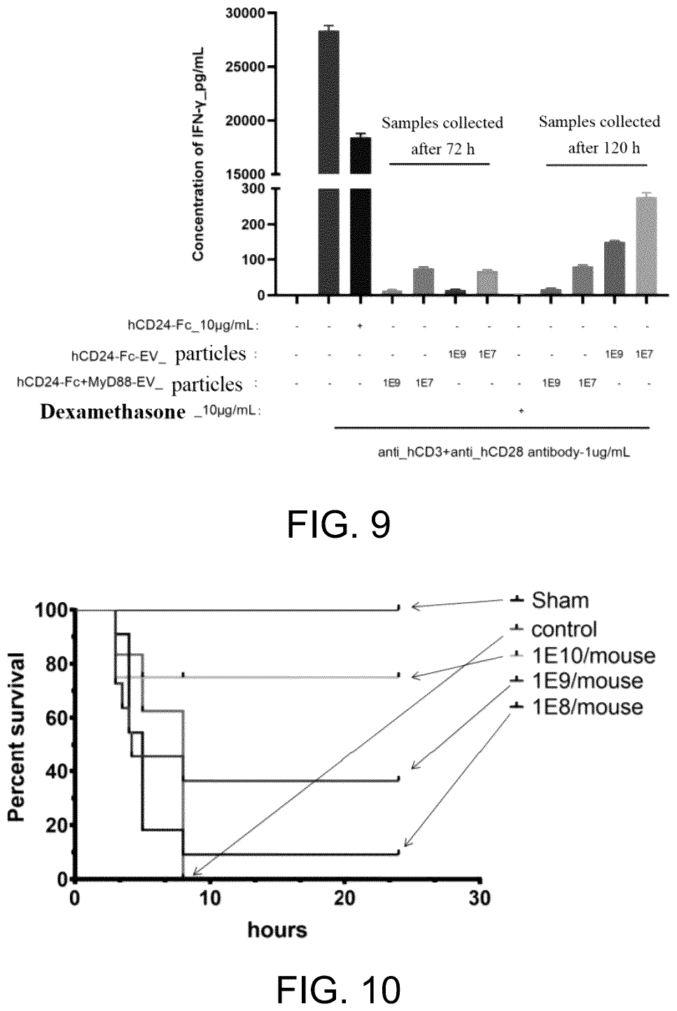

The results are shown in Tables 3-4 and in FIG. 9.

TABLE 3
Results of Loading Myd88 Inhibitor Polypeptide
into dimeric CD24 Exosomes

| | | Number of |
|---|---|---|
| | Total particle | polypeptides loaded |
| | count of | per exosome |
| | exosomes | particle |
| Sample name | loaded | (Mean ± SD) |
| dimeric CD24 Exosomes | 8.00E+11 | 0 ± 1.0 |
| dimeric CD24 Exosomes pH 8.0 + | 7.32E+11 | 37992 ± 28 |
| MyD88 Inhibitor Polypeptide | | |
| dimeric CD24 Exosomes pH 9.0 + | 6.90E+11 | 51392 ± 55 |
| MyD88 Inhibitor Polypeptide | | |
| dimeric CD24 Exosomes pH 10.0 + | 7.10E+11 | 49771 ± 40 |
| MyD88 Inhibitor Polypeptide | | |

TABLE 4
Concentration of IFN-γ in PBMC Supernatants for Different Treatment Groups

| Anti_hCD3 + | | | Corresponding | | | | | | |
|---|---|---|---|---|---|---|---|---|---|
| anti_hCD28 | Treatment | | hCD24 Protein | | Detected IFN-γ | | | IFN-γ_pg/mL- | |
| antibody_1 μg/mL | time | Treatment | Concentration | | concentration (pg/mL) | | | Mean | SD |
| − | | — | | | 0.23 | 0.56 | 0.34 | 0.4 | 0.17 |
| + | | — | | | 28325 | 28869 | 27987 | 28393.7 | 444.99 |
| | | hCD24- | | | 18210 | 18847 | 18324 | 18460.3 | 339.68 |
| | | Fc_10 μg/mL | | | | | | | |
| | 72 h | hCD24-Fc + MyD88 | 4 | μg/mL | 12 | 16 | 15 | 14.3 | 2.08 |
| | | Inhibitor | | | | | | | |
| | | Polypeptide - | | | | | | | |
| | | EV_1E9 | | | | | | | |
| | | hCD24-Fc + MyD88 | 0.04 | μg/mL | 76 | 73 | 80 | 76.3 | 3.51 |
| | | Inhibitor | | | | | | | |
| | | Polypeptide - | | | | | | | |
| | | EV_1E7 | | | | | | | |
| | | hCD24-Fc-EV_1E9 | 4 | μg/mL | 14 | 17 | 15 | 15.3 | 1.53 |
| | | hCD24-Fc-EV_1E7 | 0.04 | μg/mL | 68 | 72 | 65 | 68.3 | 3.51 |
| | | Dexamethasone_10 | | | 4.32 | 4.76 | 4.5 | 4.5 | 0.22 |
| | | μg/mL | | | | | | | |
| | 96 h | hCD24-Fc + MyD88 | 4 | μg/mL | 15.6 | 20.1 | 18.6 | 18.1 | 2.29 |

| Inhibitor | | | | | | | |
|---|---|---|---|---|---|---|---|
| Polypeptide - | | | | | | | |
| EV_1E9 | | | | | | | |
| hCD24-Fc + MyD88 | 0.04 | μg/mL | 78.6 | 82.6 | 84.5 | 81.9 | 3.01 |
| Inhibitor | | | | | | | |
| Polypeptide - | | | | | | | |
| EV_1E7 | | | | | | | |
| hCD24-Fc-EV_1E9 | 4 | μg/mL | 153 | 148 | 150 | 150.3 | 2.52 |
| hCD24-Fc-EV_1E7 | 0.04 | μg/mL | 265 | 287 | 279 | 277.0 | 11.14 |

3. Conclusion: Free ApoE proteins, dimeric CD24-loaded exosomes, and exosomes loaded with both dimeric CD24 and MyD88 inhibitor polypeptide all demonstrated better inflammation inhibitory activity in the in vitro PBMC inflammation model compared to the model group. Additionally, the exosomes loaded with both dimeric CD24 and MyD88 inhibitor polypeptide exhibited a longer duration of action.

### Example 7: Exosomes Loaded with Both Dimeric CD24 and MyD88 Inhibitor Polypeptide Significantly Reduced Mortality in a Mouse Model of Sepsis

The MyD88 inhibitor polypeptide can exert inflammation inhibition efficacy and effectively prolong the duration of drug's action. CD24-exosomes loaded with this inhibitor polypeptide were used in a mouse model of sepsis can significantly reduce mouse mortality.

### 1. Methods:

(1) Preparation of dimeric CD24 Exosomes: The plasmid construction, cell line screening and exosome preparation processes were consistent with those in Example 1.
(2) Loading of MyD88 Inhibitor Polypeptide: It was consistent with that in Example 6.
(3) Verification of Reduction Effect on Mortality and Inflammatory Factors in Blood: Eight-week-old male C57BL6 mice were selected for the experiment. A model was established by intraperitoneally injecting 10 mg/kg LPS, followed by tail vein administration of various doses of CD24-MyD88 inhibitor polypeptide-EV (each mouse receiving 1E8 to 1E10 exosome particles, with 1E9 exosomes loaded with 1 μg of protein). The mortality of experimental animals in each group were monitored and recorded at different time points over 24 hours. After 24 hours, the animals were sacrificed, and blood samples were collected to assess the levels of cytokines (IL6, IL10, and IFN-γ).

### 2. Results:

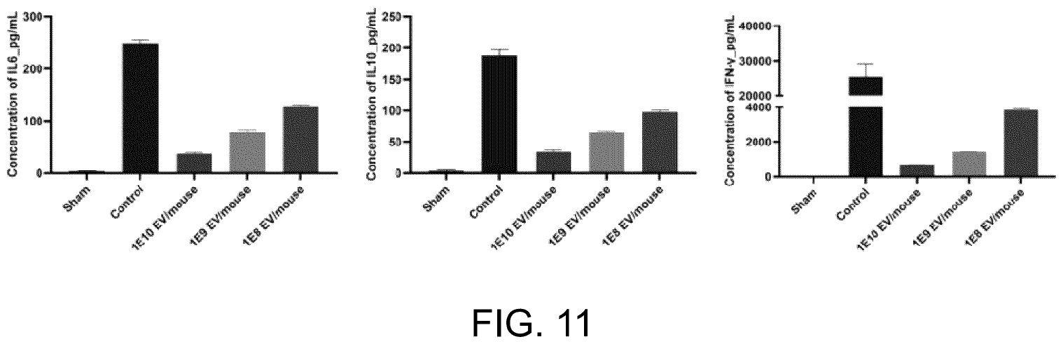

The results are shown in Tables 5-7 and FIGS. 10-11.

TABLE 5
Concentration of Cytokine IL6 in Blood of
Mice from Different Administration Groups

| | Detected IL6 Concentration | | | IL6_pg/mL- | |
|---|---|---|---|---|---|
| Treatment | (pg/mL) | | | Mean | SD |
| Sham | 4 | 3 | 3.8 | 3.6 | 0.53 |
| Control | 248 | 256 | 243 | 249.0 | 6.56 |
| 1E10 EV/mouse | 40 | 37 | 35 | 37.3 | 2.52 |
| 1E9 EV/mouse | 78 | 75 | 83 | 78.7 | 4.04 |
| 1E8 EV/mouse | 123 | 131 | 128 | 127.3 | 4.04 |

TABLE 6
Concentration of Cytokine IL10 in Blood of
Mice from Different Administration Groups

| | Detected IL10 Concentration | | | IL10_pg/mL- | |
|---|---|---|---|---|---|
| Treatment | (pg/mL) | | | Mean | SD |
| Sham | 5.1 | 2.1 | 3.6 | 3.6 | 1.46 |
| Control | 198.0 | 186.0 | 180.0 | 188.0 | 9.17 |
| 1E10 EV/mouse | 34.0 | 37.0 | 31.0 | 34.0 | 3.00 |
| 1E9 EV/mouse | 67.0 | 65.0 | 63.0 | 65.0 | 2.00 |
| 1E8 EV/mouse | 101.0 | 98.0 | 95.0 | 98.0 | 3.00 |

TABLE 7
Concentration of Cytokine IFN-γ in Blood
of Mice from Different Administration Groups

| | Detected IFN-γ Concentration | | | IFN-γ_pg/ | |
|---|---|---|---|---|---|
| Treatment | (pg/mL) | | | mL-Mean | SD |
| Sham | 5.1 | 2.1 | 3.6 | 3.6 | 1.46 |
| Control | 23507.1 | 23128.6 | 29807.1 | 25481.0 | 3751.37 |
| 1E10 EV/mouse | 674.9 | 675.1 | 701.1 | 683.7 | 15.08 |
| 1E9 EV/mouse | 1439.9 | 1421.4 | 1406.3 | 1422.5 | 16.81 |
| 1E8 EV/mouse | 3894.4 | 3773.8 | 3871.4 | 3846.6 | 64.06 |

3. Conclusion: Exosomes loaded with both dimeric CD24 and MyD88 inhibitor polypeptide can significantly reduce mouse mortality and the expression levels of inflammation-related factors (IL6, IL10, and IFN-γ) in the blood in the mouse model of sepsis.

### Example 8: Exosomes Loaded with Dimeric CD24, Dimeric ApoE and MyD88 Inhibitor Polypeptides Significantly Reduced Mortality and Protected Lung Tissue Integrity in a Mouse Model of Acute Respiratory Distress Syndrome

The MyD88 inhibitor polypeptide can exert inflammation inhibition efficacy and effectively prolong the duration of drug's action. CD24-exosomes, ApoE-exosomes, or CD24-ApoE-exosomes loaded with this inhibitor polypeptide were used in a mouse model of acute respiratory distress syndrome can reduce mouse mortality and protect lung tissue integrity.

### 1. Methods:

(1) Preparation of CD24-ApoE Exosomes: The plasmid construction, cell line screening and exosome preparation processes were consistent with those in Example 5.
(2) Loading of MyD88 Inhibitor Polypeptide: CD24-ApoE exosomes were mixed well with MyD88 inhibitor polypeptide (1 mg of MyD88 added to 1E+12 exosome particles). The solution was adjusted to a pH of 8.0 (or 9.0, 10.0) with Na₂CO₃ and then purified using Capto core700 to remove free MyD88 inhibitor polypeptide. The flow-through was collected to obtain CD24-ApoE exosomes loaded with MyD88 inhibitor polypeptide. The loading amount of MyD88 inhibitor polypeptide was assessed using ELISA.
(3) Verification of Protection Effects on Lung Tissue: Male C57BL6 mice (8-10 weeks old) were anaesthetized with isoflurane. A model was established by intraperitoneally injecting 5 mg/kg body weight of LPS, followed by tail vein injection of different types of CD24 EVs (each containing 1E9 exosome particles loaded with 1 μg of protein). The hepatoprotective effect was assessed by monitoring mortality. Lung tissue integrity was evaluated through histological examination. 5% Evans-labeled albumin was instilled into the right lower lobe of mice's lungs at a volume of 4 mL per kg body weight, and alveolar fluid clearance (AFC) was calculated 30 minutes later.

### 2. Results:

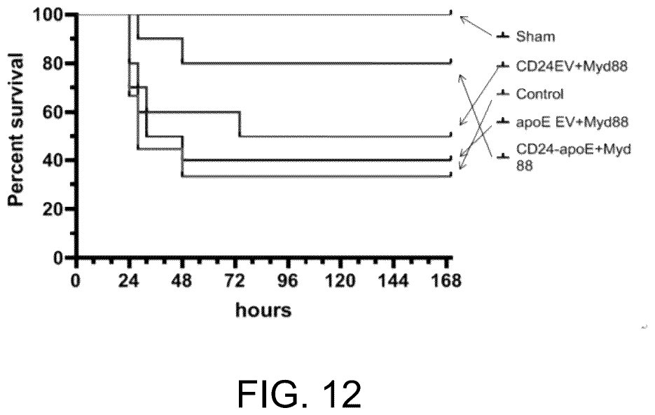

The results are shown in Table 8 and FIG. 12.

TABLE 8
Results of Loading Myd88 Inhibitor
Polypeptide into CD24-ApoE Exosomes

| | | Number of |
|---|---|---|
| | Total particle | polypeptides loaded |
| | count of | per exosome |
| | exosomes | particle |
| Sample name | loaded | (Mean ± SD) |
| CD24-ApoE Exosomes | 7.66E+11 | 0 ± 1.0 |
| CD24-ApoE Exosomes pH 8.0 + | 7.53E+11 | 37539 ± 25 |
| MyD88 Inhibitor Polypeptide | | |
| CD24-ApoE Exosomes pH 9.0 + | 7.81E+11 | 48332 ± 29 |
| MyD88 Inhibitor Polypeptide | | |
| CD24-ApoE Exosomes pH 10.0 + | 7.43E+11 | 46517 ± 36 |
| MyD88 Inhibitor Polypeptide | | |

### 3. Conclusion:

CD24-exosomes, ApoE-exosomes or CD24-ApoE-exosomes loaded with the inhibitor polypeptide can reduce mouse mortality and protect the integrity of lung tissue in a mouse model of acute respiratory distress syndrome.

The present disclosure may be outlined in other specific forms which are not contrary to the spirit or main features of the present disclosure. Accordingly, in any view of the matter, the embodiments above of the present disclosure can only be considered as exemplary only of the present disclosure and not as limiting the present disclosure. The claims define the scope of the present disclosure, whereas the foregoing description does not indicate the scope of the present disclosure, and therefore various change in meaning and scope within the equivalents of the claims are included in the scope of the claims of the present disclosure.

1. A method for loading dimeric CD24 into exosomes from HEK293 cells with ADAM10 gene knockout, comprising steps of:
(1) knocking out ADAM10 gene in HEK293 cells,
(2) loading dimeric CD24 and/or dimeric ApoE into exosomes from HEK293 cells with ADAM10 gene knockout, and
(3) verifying loading amount and/or function.

2. The method according to claim 1, wherein in step (1), the knockout of ADAM10 gene in HEK293 cells is carried out using a CRISPR gene editing system or method.

3. The method according to claim 1, wherein in step (2), the loading comprises constructing pCD24-Fc-Ig3(NPTN-Ig3)-TMD(NPTN-TMD)-ICD(EWI-F-ICD) plasmid using VB220306-1137jmq as a vector, co-transfecting with Sleeping Beauty transposase expression vector pCMV-(CAT)T7-SB100X into or infecting HEK293 cells and/or ADAM10⁻ HEK293 cells via a viral vector with the plasmid, and screening a single clone to obtain an engineered stable transgenic cell line of HEK293-CD24 or ADAM10⁻ HEK293-CD24.

4. The method according to claim 1, wherein step (2) comprises expanding and culturing an engineered stable transgenic cell line to a cell density of 5E+06 cells/mL, performing centrifugation at 5000 rpm for 30 min, and performing purification from cell supernatant to obtain engineered dimeric CD24 loaded-exosomes.

5. The method according to claim 1, wherein in step (3), the verification of loading amount is carried out by collecting cellular

6. The method according to claim 1, wherein in step (3), the verification of function is carried out by assessing a role of dimeric CD24 loaded-exosomes on mortality and liver integrity in a mouse model of acute liver failure.

7. The method according to claim 1, wherein in step (2), the loading of dimeric ApoE protein into exosomes comprises constructing a lentiviral expression vector pApoE-Fc-Ig3(NPTN-Ig3)-TMD(NPTN-TMD)-ICD(EWI-F-ICD) plasmid based on the lentiviral plasmid vector pSLenti-CMV--PGK-PuroWPRE, packaging the virus, infecting ADAM10⁻ HEK293 cells at an MOI of 10, replacing medium after 16 h and 24 h of infection, and screening a single clone to obtain an engineered stable transgenic cell line of ADAM10⁻ HEK293-ApoE.

8. (canceled)

9. The method according to claim 1, wherein step (2) comprises loading dimeric CD24 and dimeric ApoE into exosomes.

10. The method according to claim 7, wherein the loading of dimeric CD24 and dimeric ApoE protein into exosomes comprises co-transfecting into or infecting HEK293 cells via a viral vector with a constructed pApoE-Fc-Ig3(NPTN-Ig3)-TMD(NPTN-TMD)-ICD(EWI-F-ICD) plasmid and a constructed pCD24-Fc-Ig3(NPTN-Ig3)-TMD(NPTN-TMD)-ICD(EWI-F-ICD) plasmid, and screening to obtain an engineered stable transgenic cell line of ADAM10⁻ HEK293-CD24-ApoE.

11. The method according to claim 7, wherein the method further comprises expanding and culturing an engineered stable transgenic cell line to a cell density of 5E+06 cells/mL, performing centrifugation at 5000 rpm for 30 min, and performing purification from cell supernatant to obtain engineered loaded exosomes.

12. The method according to claim 11, wherein in step (3), the verification comprises detecting an immunosuppressive activity of different EVs and proteins using a PBMC inflammation model and/or establishing a mouse model of acute liver failure, and verifying a hepatoprotective effect of CD24 EV based on mortality.

13. The method according to claim 1, wherein step (2) comprises loading both dimeric CD24 and MyD88 inhibitor polypeptide into exosomes.

14. The method according to claim 13, wherein the loading of both dimeric CD24 and MyD88 inhibitor polypeptide into exosomes comprises mixing dimeric CD24 exosomes with MyD88 inhibitor polypeptide, adjusting pH to 8.0, purifying using Capto core700 to remove free MyD88 inhibitor polypeptide, and collecting a flow-through to obtain CD24 exosomes loaded with MyD88 inhibitor polypeptide.

15. The method according to claim 14, wherein the dimeric CD24 exosomes are mixed with the MyD88 inhibitor polypeptide at a ratio of 1 mg of MyD88 inhibitor polypeptide per 1E+12 exosome particles.

16. The method according to claim 13, wherein in step (3), the verification comprises detecting an immunosuppressive activity of different EVs and proteins using a PBMC inflammation model.

17. The method according to claim 13, wherein in step (3), the verification comprises detecting mortality in a mouse model of sepsis.

18. The method according to claim 1, wherein step (2) comprises loading dimeric CD24, dimeric ApoE and MyD88 inhibitor polypeptide into exosomes.

19. The method according to claim 18, wherein in step (3), the verification comprises detecting a significant reduction in mortality and protection of lung tissue integrity in a mouse model of acute respiratory distress syndrome.

20. (canceled)

21. (canceled)

22. (canceled)

23. A pharmaceutical composition, comprising an exosome produced by a method for loading dimeric CD24 into exosomes from HEK293 cells with ADAM10 gene knockout, wherein the method comprises steps of:
(1) knocking out ADAM10 gene in HEK293 cells,
(2) loading dimeric CD24 and/or dimeric ApoE into exosomes from HEK293 cells with ADAM10 gene knockout, and
(3) verifying loading amount and/or function.

24. A method of treatment, comprising administering a pharmaceutical composition comprising an exosome produced by a method for loading dimeric CD24 into exosomes from HEK293 cells with ADAM10 gene knockout, wherein the method comprises steps of:
(1) knocking out ADAM10 gene in HEK293 cells,
(2) loading dimeric CD24 and/or dimeric ApoE into exosomes from HEK293 cells with ADAM10 gene knockout, and
(3) verifying loading amount and/or function.
